(19) United States
(12) Patent Application Publication
(10) Pub. No.: US 2020/0166525 A1
(43) Pub. Date: May 28, 2020

(54) DIAGNOSIS-AIDING METHOD FOR DETERMINING NEURODEGENERATIVE DISEASE

(71) Applicants: Toru HASEGAWA, Saga (JP); NIPRO CORPORATION, Osaka (JP)

(72) Inventors: Toru HASEGAWA, Saga (JP); Hiroshi YOSHIDA, Osaka (JP); Yoshinori KOSOKU, Osaka (JP)

(73) Assignees: Toru HASEGAWA, Saga (JP); NIPRO CORPORATION, Osaka (JP)

(21) Appl. No.: 16/631,619

(22) PCT Filed: Jul. 24, 2018
(86) PCT No.: PCT/JP2018/027661

§ 371 Date: Jan. 16, 2020

(30) Foreign Application Priority Data
Jul. 25, 2017 (JP) 2017-143635

(51) Int. Cl. G01N 33/68; G01N 33/74; G16H 50/30; A61B 5/055; A61B 6/03

(52) U.S. Cl. CPC G01N 33/6896; G01N 33/74; G01N 2800/2821; A61B 5/055; A61B 6/037; G16H 50/30

(57) ABSTRACT

Provided is a method for aiding diagnosis of a neurodegenerative disease in a subject with a light physical burden. The method for aiding diagnosis of the neurodegenerative disease, comprising: a first assessment step of assessing a risk of the neurodegenerative disease based on an amount of homocysteine acid in a biological sample obtained from the subject; a second assessment step of assessing a risk of the neurodegenerative disease based on an amount of an inflammatory factor, a pituitary gland secretion, or an autonomic nerve secretion in the biological sample obtained from the subject or based on a measured value obtained from a brain image of the subject; and a step of determining whether the subject has the neurodegenerative disease based on the results of the first assessment and the second assessment.

### TECHNICAL FIELD

The invention relates to a diagnosis-aiding method for determining a neurodegenerative disease.

### BACKGROUND ART

Alzheimer's disease (also referred to as “AD” or “Alzheimer-type dementia”), dementia with Lewy bodies, vascular dementia, and frontotemporal dementia and the like are known as neurodegenerative diseases in which the cognitive function is impaired. The AD is a progressive neurodegenerative disease that causes memory impairment and dementia. In the situation where no radical therapy for AD is present, the importance of therapeutic intervention, at a stage in which there are one or more AD lesions but there is even no symptom, has been discussed for preventing dementia due to the AD.

Examples of AD diagnoses based on a biomarker include imaging examinations based on the atrophy of the medial temporal lobe. In imaging examinations performed for the AD diagnosis, nuclear magnetic resonance images (MRI) and positron emission tomography (PET) images are used.

Known biochemical biomarkers used for AD diagnoses include the decrease of amyloid-beta (Aβ) 42 or the increase of phosphorylated tau in the cerebrospinal fluid (CSF). The change in the amount of homocysteine acid in blood or in urine has been found as another biochemical biomarker (Patent Literature 1).

### CITATION LIST

### Patent Literature 1: Japanese Patent Application Publication No. 2013-253781 A

### SUMMARY OF INVENTION

An object of the invention is to provide a novel diagnosis-aiding method for a neurodegenerative disease with a light physical burden. The invention provides a highly accurate diagnosis-aiding method for a neurodegenerative disease with a light physical burden. The invention also provides a highly accurate diagnosis-aiding method for a neurodegenerative disease with a light physical burden that is any of Alzheimer-type dementia, dementia with Lewy bodies, vascular dementia, and frontotemporal dementia (hereinafter, also referred to as “Alzheimer-type dementia and the like”).

The inventors searched for biomarkers available for a diagnosis-aiding method for a neurodegenerative disease with a light physical burden. The inventors found that combining the concentration of homocysteic acid in a blood sample and the concentration of an inflammatory factor, a pituitary gland secretion, or an autonomic nerve secretion in a blood sample or a measured value obtained from a brain image (the brain volume or the area of amyloid plaques) allows highly accurate determination of whether a subject has a neurodegenerative disease, and made the invention. The inventors further found that combining the concentration of homocysteic acid in a blood sample and a measured value of a measuring factor (an inflammatory factor, a pituitary gland secretion, an autonomic nerve secretion, a brain image) in a blood sample allows determining which of Alzheimer-type dementia, dementia with Lewy bodies, vascular dementia, or frontotemporal dementia the neurodegenerative disease is classified into.

### Solution to Problem

The invention provides a method for aiding diagnosis of a neurodegenerative disease in a subject, a kit used in the method, and a method of treatment, comprising administering an agent to a subject determined to have a neurodegenerative disease in the method, as described below.

One aspect of the invention provides a method for aiding diagnosis of a neurodegenerative disease in a subject, comprising a first assessment step of assessing risk of a neurodegenerative disease based on an amount of homocysteine acid in a biological sample obtained from the subject; a second assessment step of assessing a risk of the neurodegenerative disease based on an amount of an inflammatory factor, a pituitary gland secretion, or an autonomic nerve secretion in the biological sample obtained from the subject or based on a measured value obtained from a brain image of the subject (hereinafter, referred to as a “measured value of brain image”); and a step of determining whether the subject has the neurodegenerative disease based on the results of the first assessment and the second assessment.

One aspect of the invention provides a method for aiding diagnosis of a neurodegenerative disease in a subject, comprising a step of assessing a risk of the neurodegenerative disease based on at least two measured values of measuring factors selected from the group consisting of a measured value of homocysteic acid, a measured value of an inflammatory factor, a measured value of a pituitary gland secretion, and a measured value of an autonomic nerve secretion in a biological sample obtained from the subject and a measured value obtained from a brain image of the subject; and a step of determining whether the subject has the neurodegenerative disease based on the result of the assessment; wherein the at least two measured values of the measuring factors comprise the measured value of homocysteic acid.

One aspect of the invention provides a method of treatment, further comprising a step of treating the subject based on the result of the determination in the method.

One aspect of the invention provides a kit for using in the above method, comprising a reagent for measuring at least two measuring factors selected from the group consisting of homocysteic acid, an inflammatory factor, a pituitary gland secretion, and an autonomic nerve secretion in a biological sample obtained from a subject and a brain image, wherein the reagent is a reagent for obtaining the brain image when the measuring factor is the brain image.

### Technical Effects

The invention can provide a novel diagnosis-aiding method for a neurodegenerative disease with a light physical burden. The invention can provide a highly accurate diagnosis-aiding method for a neurodegenerative disease with a light physical burden. The invention can also provide a highly accurate method with a light physical burden, for aiding diagnosing which of Alzheimer-type dementia, dementia with Lewy bodies, vascular dementia, or frontotemporal dementia a neurodegenerative disease is.

### BRIEF DESCRIPTION OF DRAWINGS

FIG. 1 is a set of graphs, where the abscissa represents the concentration of homocysteic acid (HCA) in a blood sample and the ordinate represents the concentration of a pituitary gland secretion (A) or the concentration of an inflammatory factor (B). As the concentration thereof increases, a risk of a neurodegenerative disease is assessed as higher. The graphs display the determination indicated by regions (a to d) divided with a threshold for HCA (hereinafter, referred to as the “threshold (HCA)”) on the abscissa and a threshold for a pituitary gland secretion (A) or an inflammatory factor (B) (hereinafter, referred to as the “threshold (pituitary gland secretion)” or the “threshold (inflammatory factor)”) on the ordinate. The determinations of the regions are as follows: (a): a neurodegenerative disease is suspected (A) or any of neurodegenerative diseases such as Alzheimer-type dementia is suspected (B); (b): there are no findings of a neurodegenerative disease (A, B); (c): the subject has a neurodegenerative disease (A) or the subject has any of neurodegenerative diseases such as Alzheimer-type dementia (B); and (d): a neurodegenerative disease is suspected (A, B).

FIG. 2 is a set of (A) a flow chart illustrating a diagnosis-aiding method of a neurodegenerative disease according to one embodiment performed with a detection device; (B) a flow chart illustrating the first assessment step; and (C) a flow chart illustrating the second assessment step.

FIG. 3 is a receiver operating characteristic (ROC) graph for examining the usefulness of a method for distinguishing a subject with Alzheimer's disease (AD) and a negative control (NC) subject. The abscissa represents [1-specificity] and the ordinate represents [sensitivity]. The circle (◯) in the figure indicates the point that is close in distance to the point where (1-specificity:sensitivity) is (0:1) in the ROC graph.

FIG. 4 is an ROC graph for examining the usefulness of a method for distinguishing a subject “suspected of dementia” and a subject “without any suspect of dementia or MCI”. The abscissa represents [1-specificity] and the ordinate represents [sensitivity]. The circle (◯) in the figure indicates the point that is close in distance to the point where (1-specificity:sensitivity) is (0:1) in the ROC graph.

FIG. 5 is an ROC graph for examining the usefulness of a method for distinguishing a subject “suspected of MCI” and a subject “without any suspect of dementia or MCI”. The abscissa represents [1-specificity] and the ordinate represents [sensitivity]. The circle (◯) in the figure indicates the point that is close in distance to the point where (1-specificity:sensitivity) is (0:1) in the ROC graph.

### DESCRIPTION OF EMBODIMENTS

The term “neurodegenerative diseases” as used herein means diseases in which a particular nerve cell population (for example, neural cells related to the cognitive function or cells related to the motor function) among neural cells in the brain or the spinal cord is gradually impaired and sloughed off. The neurodegenerative diseases in one embodiment are Alzheimer-type dementia, dementia with Lewy bodies, vascular dementia, and frontotemporal dementia (hereinafter, also referred to as “Alzheimer-type dementia and the like”). In one embodiment, a neurodegenerative disease is Alzheimer-type dementia. Alzheimer-type dementia is a progressive neurodegenerative disease leading to dementia.

The “subject” as used herein includes, for example, mammals and are a dog, a cow, a sheep, a nonhuman primate, and a human. In one embodiment, the subject is preferably a human. In one embodiment, the subject is a human diagnosed as having a neurodegenerative disease or a human found to have no specific sign of dementia.

The “biological sample” as used herein is not particularly limited as long as it is a biogenic sample in which a factor to be measured (for example, homocysteic acid, an inflammatory factor, a pituitary gland secretion, or an autonomic nerve secretion) can be detected, and the biological sample may be, for example, a sample derived from blood (hereinafter, referred to as a “blood sample”) or a sample derived from urine (hereinafter, referred to as a “urine sample”).

The “blood sample” as used herein is not particularly limited as long as the amount of the factor to be measured (hereinafter, also referred to as the “measuring factor”) therein can be measured, and the blood sample may be plasma, serum, or blood (for example, whole blood) collected from the subject. The plasma or serum is what tangible components such as erythrocytes have been removed from the blood. As used herein, the “urine sample” is not particularly limited as long as the amount of the measuring factor therein can be measured, and the urine sample may be urine (for example, a daily urine collection or spot urine) collected from the subject.

In one embodiment, the biological sample is a blood sample or a urine sample when the measuring factor is homocysteic acid. In one embodiment, the biological sample is a blood sample when the measuring factor is an inflammatory factor, a pituitary gland secretion, or an autonomic nerve secretion. In this example, it is convenient from the viewpoint of trouble of sampling when the biological sample for measuring homocysteic acid is a blood sample.

The “homocysteic acid (HCA)” is a kind of an amino acid known to promote the accumulation of amyloid. The amount of HCA in blood is known to increase as a neurodegenerative disease progresses. The amount of HCA in urine is known to decrease as a neurodegenerative disease progresses.

In subjects having a neurodegenerative disease, the excretion of HCA into the urine is suppressed and the concentration HCA in blood increases as symptoms thereof progress. When the concentration of HCA in blood increases, HCA passes through the blood-brain barrier and enters the brain. The HCA entered in the brain is known to function as an NMDA receptor agonist, namely a messenger that stimulates NMDA receptor. It is known that an amyloid protein produces the neurodegenerative effect in the brain when an NMDA receptor is activated.

The “inflammatory factor” as used herein means a factor whose concentration changes in association with the progression of inflammation. In the invention, a factor, among inflammatory factors, whose amount in a biological sample changes in association with the progression of a neurodegenerative disease is measured. Examples of such an inflammatory factor include, but are not limited to, tumor necrosis factor (TNF)-α, IL-1β, and C-reactive protein (CRP).

TNF-α and IL-1β are known to have a tendency of increasing in amount in blood as a neurodegenerative disease progresses. CRP is known to have a tendency of decreasing in amount in blood as a neurodegenerative disease progresses. In a particular situation, CRP is known to have a tendency of increasing in amount in blood as a neurodegenerative disease progresses. In one embodiment, the inflammatory factor is TNF-α, IL-1β, or CRP. In another embodiment, the inflammatory factor is TNF-α or CRP.

The “pituitary gland secretion” as used herein means a substance secreted from the pituitary gland for suppressing stress. Examples of the pituitary gland secretion include, but are not limited to, an adrenocorticotropic hormone (for example, cortisol) or an adrenocorticotropic hormone (ACTH). Cortisol or ACTH is known to have a tendency of increasing in amount in biological samples as a neurodegenerative disease progresses. In one embodiment, the pituitary gland secretion is cortisol or ACTH.

The “autonomic nerve secretion” as used herein means a substance that is excessively secreted when autonomic nerve ataxia progresses. As a neurodegenerative disease (for example, AD) progresses, autonomic nerve ataxia progresses and therefore the autonomic nerve secretions increase in amount. Examples of such autonomic nerve secretions include, but are not limited to, adrenaline or noradrenaline. Adrenaline is known to have a tendency of increasing in amount in CSF or blood as a neurodegenerative disease progresses. In one embodiment, the autonomic nerve secretion is adrenaline or noradrenaline.

As used herein, the amount or measured value of HCA, an inflammatory factor, a pituitary gland secretion, or an autonomic nerve secretion in a biological sample is not particularly limited as long as it is a value that can be quantitatively assessed, and may be, for example, the concentration or content of the substance per 1 ml of the biological sample. The amount or measured value in a biological sample can be quantitatively measured by a routine method. Examples of such a method include, but are not limited to, ELISA using or combined with the antigen-antibody reaction and liquid chromatography (for example, HPLC). In one embodiment, the amount or measured value of HCA, an inflammatory factor, a pituitary gland secretion, or an autonomic nerve secretion in a biological sample is measured by ELISA.

In another examples, the amount or measured value of HCA, an inflammatory factor, a pituitary gland secretion, or an autonomic nerve secretion in a biological sample may be a measurement result (relative value) of a parameter obtained by the measuring method. When a measuring method is fluorescence ELISA, the amount or measured value in a biological sample may be the fluorescence intensity obtained as a result of fluorescence ELISA. When the amount or measured value of a substance in a biological sample is measured using fluorescence ELISA, a standard sample containing the corresponding substance with a known concentration may also be measured using fluorescence ELISA. In this case, the assessment step described later may be conducted by comparing the fluorescence intensity from the biological sample with the fluorescence intensity (threshold) from the standard sample.

The “brain image” as used herein means an image obtained by imaging the brain of the subject alive. Examples of the brain image include, but are not limited to, an MRI image, a PET image, and a CT image.

In one embodiment, the PET image is an image obtaining by PET imaging amyloid plaques, as a marker, that accumulate as AD progresses (hereinafter, also referred to as “amyloid PET”). Examples of a PET tracer used in the amyloid imaging include, but are not limited to, ¹¹C—PiB.

The “measured value obtained from a brain image” as used herein (also referred to as the “measured value of brain image”) means a value measured from a particular region of the brain image. In one embodiment, the measured value of brain image is the volume of the brain region whose structure is considered to change as a neurodegenerative disease (for example, AD) progresses or the area of a region that reflects the change in the accumulated amount of a particular substance (for example, amyloid peptide, tau protein).

Examples of the volume of the brain region are the volume of a particular structure (for example, the medial temporal region and the entorhinal cortex) or the volume of the whole brain obtained from an MRI image. Examples of the area of the brain region include the area of amyloid plaques obtained from the amyloid PET image. Examples of the method for calculating the measurement from an image include use of a software for automated volumetry (for example, FreeSurfer (http://surfer.nmr.mgh.harvard.edu)) and a software for the measured value of cerebral atrophy (for example, Voxel-based Specific Regional analysis system for Alzheimer's Disease: VSRAD (R)).

As used herein, the threshold of HCA, an inflammatory factor, a pituitary gland secretion, and an autonomic nerve secretion may be denoted as the threshold (HCA), the threshold (inflammatory factor), the threshold (pituitary gland secretion), and the threshold (autonomic nerve secretion), respectively. Each threshold may be a value set to assess a risk of suffering from or developing a neurodegenerative disease based on an amount of each factor in a biological sample (for example, a blood sample or an urine sample). Each threshold may be a value predetermined based on the amounts of each factor in biological samples in a neurodegenerative disease group and a non-neurodegenerative disease group or an AD group and a non-AD group. Alternatively, each threshold may be a result of measurement (relative value) of each factor from the subject before the progress of a certain period of time.

As used herein, the threshold of the measured value of brain image may be denoted as the threshold (measured value of brain image). The threshold is a value set to assess a risk of suffering from or developing a neurodegenerative disease based on a measured value. The threshold is not limited, but may be a value predetermined from a measured value obtained from brain images from a neurodegenerative disease group and a non-neurodegenerative disease group or an AD group and a non-AD group (for example, the brain volume or the area of amyloid plaques). Alternatively, the threshold may be a measured value of brain image of the subject before the progress of a certain period of time.

The “neurodegenerative disease group” or “AD group” refers to a group of subjects having a neurodegenerative disease or to a group of subjects having AD. The “non-neurodegenerative disease group” or “non-AD group” refers to a group of subjects having neither neurodegenerative disease nor AD. The “mild cognitive impairment group” refers to a group of subjects having mild cognitive impairment (MCI). The “non-mild cognitive impairment group” refers to a group of subjects not having MCI. For example, the non-AD group, the non-neurodegenerative disease group, the non-mild cognitive impairment group may each be, for example, a healthy group. The “healthy group” refers to a group of subjects selected from healthy subjects according to certain exclusion criteria. The certain exclusion criteria may include, for example, exhibiting no sign of dementia. The scale of each group in the determination of a threshold is set by a person skilled in the art as appropriate in consideration of factors such as the sensitivity, specificity, cost, and the like of the diagnosis.

In the invention, the “assessment” can be semiautomatically or automatically or mechanically made without the judgment of a person having technical knowledge, such as a physician or a laboratory technician. The “assessment” in the diagnosis-aiding method according to one embodiment of the invention includes the first assessment based on the amount of HCA in a biological sample; and/or the second assessment based on the amount of an inflammatory factor, a pituitary gland secretion, or an autonomic nerve secretion in the biological sample or a measured value of brain image.

The first assessment and/or the second assessment is semiautomatically or automatically or mechanically made by comparing the amount of each factor measured in the biological sample with the “threshold” of the factor. When the factor is, for example, a factor whose increase in the biological sample leads to a judgement that there is an increased risk of a neurodegenerative disease, then it can be assessed semiautomatically or automatically or mechanically by a person even without any technical knowledge whether the subject has or does not have a risk of suffering from or developing a neurodegenerative disease (for example, AD) based on the case where the measured value is higher or lower than the threshold.

In one embodiment, the first assessment when the biological sample is a blood sample involves comparing the amount of homocysteine acid in the blood sample with the predetermined corresponding threshold (HCA) and assessing that there is a risk of the neurodegenerative disease when the amount is larger than the threshold (HCA). In another embodiment, the first assessment when the biological sample is a urine sample involves comparing the amount of homocysteine acid in the urine sample with the predetermined corresponding threshold (HCA) and assessing that there is a risk of the neurodegenerative disease when the amount is smaller than the threshold (HCA).

In one embodiment, the second assessment comprises comparing the amount of an inflammatory factor, a pituitary gland secretion, or an autonomic nerve secretion in the biological sample with a predetermined corresponding threshold and assessing a risk of the neurodegenerative disease based on the result of the comparison. In another embodiment, the second assessment comprises comparing a measured value of brain image with a predetermined threshold (measured value of brain image) and assessing a risk of the neurodegenerative disease based on the result of the comparison.

In one embodiment, in the case where the measuring factor is an MRI brain image, the second assessment involves the assessment that there is a risk of the neurodegenerative disease when the brain volume obtained from the image is smaller than a predetermined corresponding threshold. In another embodiment, in the case where the brain image is, for example, an amyloid PET image, the second assessment involves the assessment that there is a risk of the neurodegenerative disease when the area of amyloid plaques obtained from the image is larger than a predetermined corresponding threshold.

The “assessment” in the diagnosis-aiding method according to another embodiments comprises assessing a risk of a neurodegenerative disease based on at least two measured values of measuring factors selected from the group consisting of a measured value of homocysteic acid, a measured value of an inflammatory factor, a measured value of a pituitary gland secretion, and a measured value of an autonomic nerve secretion in a biological sample and a measured value obtained from a brain image of the subject.

In the aforementioned embodiment, the assessment of the risk of the neurodegenerative disease based on the at least two measured values of measuring factors comprises, but is not limited to, calculating an assessment value from the at least two measured values of the measuring factors. The “assessment value” is, for example, calculated from a value obtained by multiplying at least two measured values of measuring factors by coefficients corresponding thereto. The coefficients by which the at least two measured values of measuring factors are multiplied are set depending on the kind of the measuring factors, the number of measuring factors, the kind (for example, weight, concentration) of the measured values as appropriate. In one embodiment, any of the at least two measured values of measuring factors is the concentration thereof in the biological sample.

The “coefficient” by which the measured value of each measuring factor is multiplied can be set, for example, by obtaining, for each subject, the sum in a linear expression of the at least two measured values of measuring factors obtained from a group of subjects diagnosed whether they have a neurodegenerative disease or not (when the measuring factors are the homocysteic acid (HCA) concentration (=[HCA]) and the TNFα concentration (=[TNFα]), the sum in a linear expression can be expressed as a×[HCA]+b×[TNFα]+c and, wherein a, b, and c are “coefficients” (a, b≠0)); making a receiver operating characteristic (ROC) curve from the obtained sums in the linear expression and the results of diagnosis of the subjects; and adjusting the values of the coefficients (a, b, c) in the linear expression so that the area under the made ROC curve (Area under the Curve: AUC) becomes a maximum. The setting of such coefficients can be made by using commercially available software (for example, ORIGIN (R) PRO9.1).

In this example, the “assessment value” can be obtained by calculating the sum in a linear expression (i.e., a×[HCA]+b×[TNFα]+c) obtained by multiplying the corresponding measuring factors by the set coefficient.

Although the at least two measuring factors are two of HCA and TNFα in the aforementioned example, the at least two measuring factors are not limited thereto and may be 3 measuring factors (for example, HCA, cortisol, and TNFα), 4 measuring factors, or a combination of 2 other measuring factors (for example, HCA and cortisol). Although, the coefficients are those adjusted so that AUC becomes the maximum in the example, the way to set coefficients is not limited thereto and the coefficients may be adjusted so that AUC exceeds a particular value.

In an example, when the measured values of at least two measuring factors are the measured value (concentration) of homocysteic acid, the measured value (concentration) of an inflammatory factor (for example, TNFα), and the measured value (concentration) of a pituitary gland secretion (for example, cortisol) in a biological sample, the ratio of the coefficients by which respective measuring factors are multiplied is, but not limited to, 1:0.005 to 0.035:4 to 11, preferably 1:0.01 to 0.03:5 to 10, and more preferably 1:0.015 to 0.025:6 to 9.

In another example, when the at least two measured values of measuring factors are the measured value (concentration) of homocysteic acid and the measured value (concentration) of an inflammatory factor (for example, TNF α) in a biological sample, the ratio of coefficients by which respective measuring factors are multiplied is, but not limited to, 1:0.5 to 3, preferably 1:0.8 to 2.5, and more preferably 1:1 to 2.

The threshold of the assessment value as used herein may be denoted as the threshold (assessment value). The threshold (assessment value) is a value set to assess a risk of suffering from or developing a neurodegenerative disease based on the at least two measured values of the measuring factors. The threshold (assessment value) is, but not limited to, a value predetermined to distinguish whether the subject has a neurodegenerative disease or non-neurodegenerative disease, AD or non-AD, or mild cognitive impairment or non-mild cognitive impairment, when compared with an assessment value calculated from the at least two measured values of the measuring factors of a subject.

In one embodiment, the threshold (assessment value) is set based on a receiver operating characteristic (ROC) graph or ROC curve. The ROC graph or ROC curve can be made according to a routine method by setting the threshold of the assessment value for the subject and calculating the sensitivity and/or the specificity with the threshold. Methods for setting the threshold (assessment value) from the ROC graph or ROC curve are not limited, but may include those involving setting based on the balance between the sensitivity and the specificity of the diagnosis-aiding method.

In one example, the threshold (assessment value) is set so that the sensitivity and/or the specificity of the diagnosis-aiding method becomes equal to or higher than a desired value (for example, the sensitivity and the specificity are both 70% or more, 75% or more, or 80% or more). In another example, the threshold (assessment value) is set to the point on the ROC graph where (1-specificity:sensitivity) is close in distance to the point (0:1) in the ROC graph. In another example, the threshold (assessment value) is set by a method using Youden index. As used herein, “Youden index” means the maximal value of (sensitivity+specificity-1).

The “sensitivity” as used herein means a quantitative indicator that indicates whether a subject having a disease (positive subject) can correctly be judged positive in a diagnosis-aiding method. For example, the sensitivity is the ratio of subjects judged positive in a group of positive subjects (=[subjects judged positive (number)]/[group of positive subjects (number)]) by a diagnosis-aiding method.

The “specificity” as used herein means a quantitative indicator that indicates whether a subject not having a disease (negative subject) can correctly be judged negative in a diagnosis-aiding method. For example, the specificity is the ratio of subjects judged negative in a group of negative subjects (=[subjects judged negative (number)]/[group of negative subjects (number)]) by a diagnosis-aiding method.

Although the expression of result of assessment in the first assessment step, the second assessment step, or the assessment step based on an assessment value is “having risk/no risk” suffering from or developing a neurodegenerative disease or MCI in the example, the diagnosis-aiding method according to the invention is not limited thereto. The expression of result of assessment may be set as appropriate and may be, for example, that the subject has “possibility/no possibility” of having a neurodegenerative disease or MCI.

In one embodiment, “determination” can be semiautomatically or automatically or mechanically made with reference to an association table of assessment result and determination preset for combinations of results of the first assessment and the second assessment. In one embodiment, when both of the results of the first assessment and the second assessment are “there is the risk” of the neurodegenerative disease, the determination that the subject “has a neurodegenerative disease” is made according to a particular association table. In one embodiment, when either one of the results of the first assessment and the second assessment is “there is a risk” of the neurodegenerative disease, the determination that the subject is “suspected of the neurodegenerative disease” is made according to a particular association table. In one embodiment, when both of the results of the first assessment and the second assessment are “there is no risk” of the neurodegenerative disease, the determination that “there are no findings” of the neurodegenerative disease is made according to a particular association table.

In another embodiment, the “determination” can be semiautomatically or automatically or mechanically made by comparing the assessment value obtained from the at least two measured values of the measuring factors with the threshold (assessment value) corresponding thereto. In one embodiment, when the assessment value is larger than the threshold (assessment value) corresponding thereto, the determination that the subject “has a neurodegenerative disease” or “has MCI” is made. In another example, when the assessment value is larger than the threshold (assessment value) corresponding thereto, the determination that the subject is “suspected of the neurodegenerative disease” or “suspected of MCI” is made. In another embodiment, when the assessment value is equal to or less than the threshold (assessment value) corresponding thereto, the determination is made that the subject “has no neurodegenerative disease” or “has no MCI”.

Although the expression of the determination is “having a neurodegenerative disease” or “having MCI” in the aforementioned example, the diagnosis-aiding method according to the invention is not limited thereto. The expression of the determination may be set as appropriate and may be, for example, that the subject is “likely” to have a neurodegenerative disease. In another example, although the expression of the determination is that a subject is “suspected of the neurodegenerative disease” or “suspected of MCI”, the diagnosis-aiding method according to the invention is not limited thereto. The expression of the determination may be, for example, “the continuation of examination is recommended”.

In one embodiment, the diagnosis-aiding method comprises determining which of Alzheimer-type dementia, dementia with Lewy bodies, vascular dementia, or frontotemporal dementia the neurodegenerative disease is classified into based on the results of the first assessment and the second assessment. In this example, the expression of the determination may be that the neurodegenerative disease “is Alzheimer-type dementia, dementia with Lewy bodies, vascular dementia, or frontotemporal dementia” or “is any of neurodegenerative diseases such as Alzheimer-type dementia”.

In this example, the classification of the neurodegenerative disease is not limited, but may be made based on the kind of the measuring factors (for example, an inflammatory factor, a pituitary gland secretion, an autonomic nerve secretion, or a brain image). In one embodiment, the classification of the neurodegenerative disease may be made based on the kind of the inflammatory factor.

In one embodiment, the second assessment in the diagnosis-aiding method comprises a step of making an assessment based on the measured value of brain image and the determining step comprises determining whether the neurodegenerative disease is classified into frontotemporal dementia based on the results of the first assessment and the second assessment. In this example, the expression of the determination may be that the neurodegenerative disease “is frontotemporal dementia”.

In one embodiment, the diagnosis-aiding method comprises determining the progression of the neurodegenerative disease using the amounts of each factor in the biological samples or measured value of brain images at least two different points in measurement time. In this example, the thresholds in the first assessment and the second assessment are the amount of each factor in a biological sample or a measured value of brain image before the progress of a certain period of time and the assessment that “there is a tendency/no tendency of aggravation” can be semiautomatically or automatically or mechanically made by comparing each of the thresholds with the corresponding amount of factor or measured value of brain image measured after the progress of a certain period of time. When the factor or brain image is, for example, a factor or brain image whose increase in amount of the biological sample or measured value of brain image leads to an assessment that there is increased risk of a neurodegenerative disease, then the assessment can be semiautomatically or automatically or mechanically made by a person even without any technical knowledge that there is a tendency or no tendency of aggravation the neurodegenerative disease in the subject based on the case where the amount of the factor or the measured value of brain image is higher or lower than the threshold.

In the determining step in this example, for example, when both of the result of the assessment just before the determination and the result of the assessment before that are that “there is a tendency of aggravation” of the neurodegenerative disease, the determination is made that the neurodegenerative disease is “progressive”. In another embodiment, when either of the result of the assessment just before the determination and the result of the assessment before that is that “there is a tendency of aggravation” of the neurodegenerative disease, the determination is made that “the continuation of examination is recommended”. In one embodiment, when both of the result of the assessment just before the determination and the result of the assessment before that are that “there is no tendency of aggravation” of the neurodegenerative disease, the determination is made that the neurodegenerative disease is “no progressive”. The determining step relating to this example can be rephrased as a step of determining the progression of a neurodegenerative disease in a subject based on the results of the first assessment and the second assessment.

In the aforementioned embodiment, the threshold (assessment values) corresponding to the assessment value is a measured value of each factor in a biological sample or a measured value of brain image before the progress of a certain period of time and the assessment that “there is a tendency/no tendency of aggravation” can be semiautomatically or automatically or mechanically made by comparing the threshold (assessment values) with the corresponding measured value of each factor or measured value of brain image measured after the progress of a certain period of time. In the determining step in this example, when both of the result of the assessment just before the determination and the result of the assessment before that are that “there is a tendency of aggravation” of the neurodegenerative disease, the determination is made that the neurodegenerative disease is “progressive”. In another example, when either of the result of the most recent assessment and the result of the assessment before that is that “there is a tendency of aggravation” of the neurodegenerative disease, the determination is made that “the continuation of examination is recommended”. In another example, when both of the result of the most recent assessment and the result of the assessment before that are that “there is no tendency of aggravation” of the neurodegenerative disease, the determination is made that the neurodegenerative disease is “no progressive”. The determining step relating to this example can be rephrased as a step of determining the progression of a neurodegenerative disease in a subject based on the result of the most recent assessment and the result of the assessment before that.

In one embodiment, the diagnosis-aiding method according to the invention may comprise a step of measuring the amount of HCA in a biological sample derived from blood obtained from a subject and the amount of an inflammatory factor, a pituitary gland secretion, or an autonomic nerve secretion in the biological sample.

When HCA in a blood sample is measured, in an example, the method comprises forming an assembly of HCA and a detection reagent therefor and detecting a signal reflecting the amount of the factor derived from the assembly. In another embodiment, the measuring step further comprises calculating the amount of the factor from the detected signal.

In one embodiment, at least two measured values of measuring factors selected from the group consisting of a measured value of homocysteic acid, a measured value of an inflammatory factor, a measured value of a pituitary gland secretion and a measured value of an autonomic nerve secretion in a biological sample, and a measured value obtained from a brain image of the subject may be, but not limited to, those obtained simultaneously or at different time points.

The “detection reagent” for each measuring factor such as HCA and an inflammatory factor comprises a “probe” that is capable of binding specifically to a measuring factor of interest. Examples of the probe include antibodies and compounds to a particular measuring factor. Examples of the antibodies include, but are not limited to, intact antibodies (for example, monoclonal antibodies), antibody fragments (for example, Fab), and synthetic antibodies (for example, chimeric antibodies). The antibodies can be prepared according to a known method, for example, an immunological technique, a phage display, or a ribosomal display. The antibodies include commercially available antibodies, which may be used itself as a probe. Examples of the compounds include substances that are capable of binding specifically to a particular measuring factor, for example, aptamers. The probe may be in a free form or be immobilized on a carrier such as beads and a plate.

The detection reagent may further comprise a “label substance” that emits signal, in addition to the probe. Examples of the label substance include fluorescent substances and enzymes. As the fluorescent substances and enzymes, known substances can be used without particular limitation and they may be commercially available. Fluorescent substances and enzymes can, for example, be produced according to a known method. When an enzyme is used as a label substance, the detection reagent comprises a substrate corresponding to the enzyme. Examples of the substrate include chromogenic substrates and chemiluminescence substrates. The label substance may be in a conjugation form which is in advance conjugated to a probe. The conjugation of the label substance may be achieved through a direct binding to the probe or an indirect linking to the probe via at least one different substance.

By placing a particular measuring factor in a blood sample and a detection reagent containing a probe therefor under a condition in which both can contact with each other, the “assembly” of the factor and the detection reagent are formed. The assembly may be separated from the unreacted particular measuring factor and detection reagent (B/F separation). When the detection reagent includes a label substance, the signal reflecting the amount of a particular measuring factor included in the assembly can be emitted from the label substance. When the assembly is formed, for example, depending on (for example, in proportion to) the amount of the particular measuring factor in the blood sample, the intensity of the signal can reflect the amount of the particular measuring factor in the blood sample. The amount in the blood sample of the factor can be calculated from the obtained signal intensity (relative value). The comparison of the amount of each calculated factor and the threshold of the factor allows the assessment of whether the donor (subject) of the measured blood sample is at risk of the neurodegenerative disease. In the above example, although the blood sample is used as a biological sample, the invention is not limited thereto.

In one embodiment, the diagnosis-aiding method may further comprise a step of treating the subject according to the result of the determination. One aspect of the invention provides a method of treatment, further comprising a step of treating the subject based on the result of the determination in the diagnosis-aiding method. The treatment step is not limited, but comprises a step of administering an agent selected according to the result of the determination (for example, the neurodegenerative disease is “AD” or “MCI”). The agent according to the result of the determination is commercially available or can be produced according to a known method. The agent can be administered in a well-known dosage and/or dose.

In one embodiment, the diagnosis-aiding method may be carried out using a diagnosis-aiding kit. One aspect of the invention provides a diagnosis-aiding kit to be used in the diagnosis-aiding method according to the invention. The diagnosis-aiding kit comprises, for example, a reagent for measuring at least two measuring factors selected from the group consisting of homocysteic acid, an inflammatory factor, a pituitary gland secretion, and an autonomic nerve secretion in a biological sample obtained from a subject and a brain image, wherein the reagent is a reagent for obtaining the brain image when the measuring factor is the brain image. The reagent may comprise a detection reagent, a probe, and/or a label substance corresponding to each measuring factor and a buffer, a cleaner, a color coupler, and the like. The kit can be produced according to a well-known method.

In another embodiment, the diagnosis-aiding method may comprise a step of generating a brain image of the subject. The method of generating the brain image is selected as appropriate by a person skilled in the art according to the kind of the brain image to be obtained. Examples of the method of generating the brain image include, but are not limited to, nuclear magnetic resonance spectroscopy, positron emission tomography (PET), and computed tomography (CT). For example, when an MRI brain image is obtained, nuclear magnetic resonance imaging (MRI) is used.

In one embodiment, the diagnosis-aiding method can be carried out with diagnostic apparatus. For example, such a diagnostic apparatus comprises a controller that controls overall operations thereof, an input unit with which a user conducts input, a displaying unit that performs screen display, and a storage device in which a database is stored. The diagnostic apparatus is connected to a measuring unit via an interface.

The controller may be composed of a processing circuit corresponding to a processor such as CPU and a memory (main storage device). The processor of the controller executes a memory-loaded computer program. The controller can realize an assessing unit and determining unit described below by executing a predetermined computer program.

The storage device is an auxiliary storage and may be, for example, a hard disk drive (HDD). In the storage device, a computer program is stored. The computer program comprises an operating system and an application program. The application program comprises an assessment program that functionalizes an assessment function and a determination program that functionalizes a determination function described below.

The database stored in the storage device may comprise threshold data (threshold (HCA), threshold (inflammatory factor), threshold (pituitary gland secretion), threshold (autonomic nerve secretion), and threshold (measured value of brain image)). The threshold data comprises a threshold for distinguishing a group having a risk of a neurodegenerative disease and a group having no risk thereof. The thresholds may be divided into subgroups based on characteristics of the subjects (for example, sex, age, race, and region), the kind of the measuring factor or measurement sample (for example, blood and urine samples). For example, the thresholds may be divided into subgroups based on the age (less than 65 years old, over 65 years old). The measurement data given identification information (for example, the full name, age, and/or ID) may be further stored in the storage device.

When a database is stored in a recording medium such as an optical disk or flash memory, the storage device may be composed of a drive device that reads and/or writes information on the recording medium and the recording medium.

The assessment unit realized by the controller has a first assessment function and a second assessment function. The assessment functions are realized by realizing the assessment programs loaded on the memory of the controller in the processing circuit including the processor of the controller.

The assessment program includes a first assessment program. The first assessment program includes the HCA concentration that a user input or an HCA concentration-obtaining program that obtains the HCA concentration based on identification information when the concentration is stored in the database; and a first threshold-obtaining program that obtains the threshold (HCA) stored in the database according to the identification information.

The assessment program further includes the second assessment program. The second assessment program includes an blood-concentration-and-the-like obtaining program that obtains the blood concentration of an inflammatory factor, a pituitary gland secretion, an autonomic nerve secretion or a measured value of brain image that a user inputs, or obtains the blood concentration or the measured value of brain image when it is stored in the database according to the identification information; and a second threshold-obtaining program that obtains the threshold (inflammatory factor), the threshold (pituitary gland secretion), the threshold (autonomic nerve secretion) or the threshold (measured value of brain image) stored in the database according to identification information.

The second assessment program involves comparing the blood concentration of an inflammatory factor, a pituitary gland secretion, or an autonomic nerve secretion or a measured value of brain image with a corresponding threshold and making the assessment whether there is a risk of the neurodegenerative disease. The second assessment program involves comparing, for example, the blood concentration of an autonomic nerve secretion with the threshold (autonomic nerve secretion), wherein increase or decrease of the concentration of the autonomic nerve secretion leads to the assessment whether there is an increased risk of the neurodegenerative disease, and making the assessment that there is a risk of the neurodegenerative disease when the concentration is larger or smaller than the threshold (autonomic nerve secretion).

The determination unit realized by the controller is realized by executing the determination program loaded on the memory of the controller in the processing circuit including the processor of the controller.

The determination program involves determining whether the subject has the neurodegenerative disease based on the results of the first assessment and the second assessment. The determination program involves determining that the subject has the neurodegenerative disease when both of the results of the first assessment and the second assessment are “there is the risk” of the neurodegenerative disease. The determination program involves determining the subject is suspected to have the neurodegenerative disease when either of the results of the first assessment and the second assessment is “there is the risk” of the neurodegenerative disease. Alternatively, the determination program involves determining that there are no findings of the neurodegenerative disease when both of the results of the first assessment and the second assessment are “there is no risk” of the neurodegenerative disease.

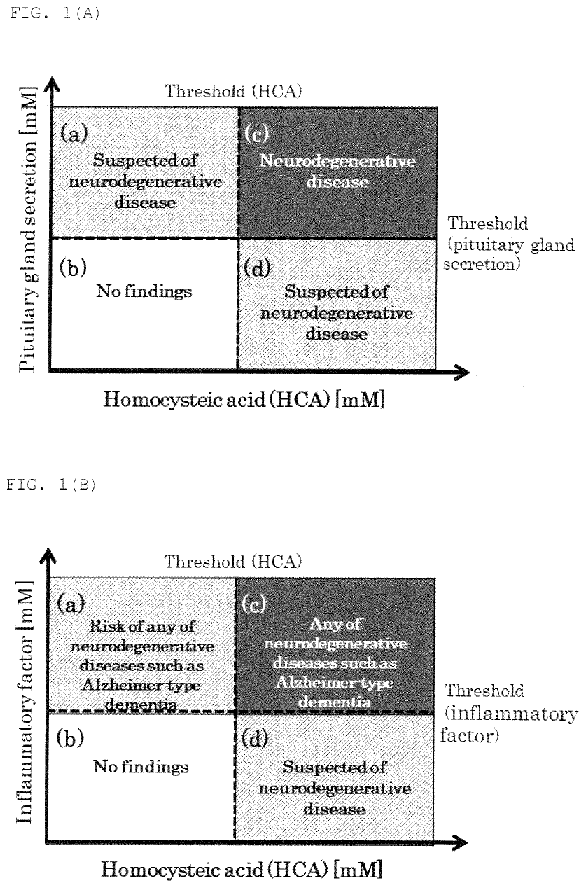

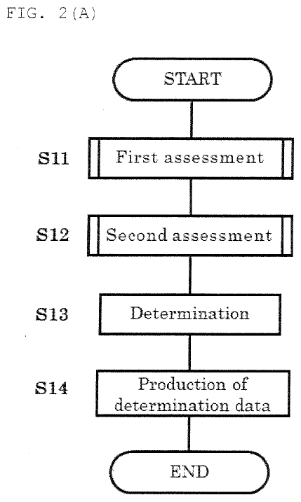

In one embodiment, in the case where the second assessment is an assessment based on the blood concentration of the pituitary gland secretion, the determination program involves determining that the subject has the neurodegenerative disease when both of the results of the first assessment and the second assessment are “there is the risk” of the neurodegenerative disease (FIG. 1(A), c). In this example, the determination program involves determining that the subject is suspected of the neurodegenerative disease when either of the results of the first assessment and the second assessment is “there is the risk” of the neurodegenerative disease (FIG. 1(A), a, d). Moreover, the determination program involves determining that there are no findings of the neurodegenerative disease when both of the results of the first assessment and the second assessment are “there is no risk” of the neurodegenerative disease (FIG. 1(A), b).

In another embodiment, in the case where the second assessment is an assessment based on the blood concentration of the inflammatory factor, the determination program involves determining that the subject has any of neurodegenerative diseases such as Alzheimer-type dementia, when both of the results of the first assessment and the second assessment are that “there is the risk” of the neurodegenerative disease (FIG. 1(B), c). In this example, the determination program involves determining that the subject is suspected of any of neurodegenerative diseases such as Alzheimer-type dementia, when the result of the first assessment is that there “is no risk” and the result of the second assessment is that there “is a risk” (FIG. 1(B), a). The determination program involves determining that the subject is suspected to have the neurodegenerative disease, when the first assessment result is that “there is a risk” and the second assessment result is that “there is no risk” (FIG. 1(B), d). Moreover, the determination program involves determining that there are no findings of the neurodegenerative disease, when both of the results of the first assessment and the second assessment are “there is no risk” of the neurodegenerative disease (FIG. 1(B), b).

The input unit is composed of equipment or a device for a user to input, besides identification information, other required information (including, for example, the kind of the measuring factor, the kind of the biological sample (for example, a blood sample or an urine sample), or the kind of the brain image) and instructions into a controller. The input unit may be, for example, a keyboard, a mouse, and a speech recognition device.

The displaying unit is composed of a device capable of allowing a user to perceive the result of determination from a determination unit and may be, for example, a display and a printer. The displaying function to a displaying unit is realized by executing an application program including a displaying program loaded on the memory of the controller in the processing circuit including the processor of the controller.

The measuring unit is composed of a device for measuring the amount of HCA, an inflammatory factor, a pituitary gland secretion, or an autonomic nerve secretion in a biological sample or a device for generating a brain image. The measuring unit may be a device and/or a kit for performing, for example, ELISA. The device for generating a brain image may be a nuclear magnetic resonance imaging (MRI) device or a positron emission tomography (PET) device. The measuring unit may include a computer for calculating the brain volume or the area of a particular factor (for example, amyloid plaques) from the brain image. The measuring unit has a function of outputting measurement data to a controller via an interface.

Although the database is stored in the storage device in the diagnostic apparatus in the aforementioned example, the diagnostic apparatus for carrying out the diagnosis-aiding method according to the invention is not limited thereto. The database may be stored, for example, in a storage device out of the diagnostic apparatus. Such a storage device may be composed, for example, by all or part of the recording medium such as optical disks. The storage device may be provided in a server connected via a network to the diagnostic apparatus according to the invention.

The diagnostic apparatus may be connected to an external measuring unit or a measuring unit may be in the diagnostic apparatus. Alternatively, a controller may be made read the measurement data obtained using the measuring unit not connected to the diagnostic apparatus via a recording medium.

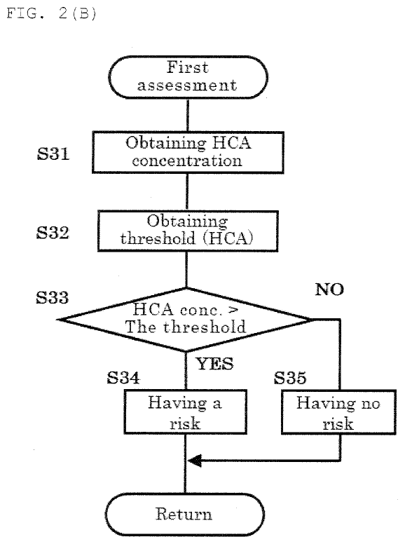

The operations of the diagnostic apparatus carrying out the diagnosis-aiding method according to one embodiment are described referring to FIG. 2.

The instructions for the controller (including the instruction of starting the diagnosis) and the information required in addition to identification information (including the designation of the diagnosis mode and a designating part that designates the database in which HCA measurement data of subjects is stored) are input by a user using an input unit. In one embodiment, the diagnostic apparatus comprises a diagnosis mode in which it is determined whether the subject has a neurodegenerative disease and a diagnosis mode in which it is determined which of those such as the Alzheimer-type dementia the neurodegenerative disease is classified into. In this example, the diagnosis mode in which it is determined whether the subject has a neurodegenerative disease is designated by the user. The processor of the controller makes the first assessment when there are input of instructions and necessary information from a user via the input unit (FIG. 2(A), S11).

In the first assessment (S11), the processor executes the first assessment program (including an HCA concentration-obtaining program and a first threshold-obtaining program) composing an assessment program loaded onto a memory from a storage device, thereby realizing an assessment unit in a controller.

FIG. 2(B) is a flow chart illustrating the first assessment. The performed assessment program obtains, referring to user-input “identification information” and the “designating part that designates a database in which HCA measurement data of subjects is stored”, the HCA concentration of the subject corresponding to the identification information from the database (S31). The performed first assessment program obtains, referring to user-input identification information, the corresponding threshold (HCA) from the threshold data stored in the database (S32).

The performed first assessment program compares the HCA concentration with the threshold (HCA) corresponding thereto (S33) and performs the first assessment. In step S33, the assessment that there is a risk of the neurodegenerative disease is made (S34) when the HCA concentration is higher than the threshold (HCA) or the assessment that there is no risk of the neurodegenerative disease is made (S35) when the HCA concentration is lower than the threshold (HCA).

If the first assessment result is obtained, then the second assessment is performed (FIG. 2(A), S12). In the second assessment (S12), the processor executes the second assessment program (including a blood concentration and the like-obtaining program and a second threshold-obtaining program) composing an assessment program loaded onto a memory from a storage device, thereby realizing an assessment unit in a controller.

FIG. 2(C) is a flow chart illustrating the second assessment. The performed second assessment program obtains, referring to user-input “identification information” and a “designating part that designates a database in which measurement data relating to the kind of measuring factor (an inflammatory factor, a pituitary gland secretion, an autonomic nerve secretion, or a brain image) is stored”, a corresponding measured value from the database (S51). In this example, the blood concentration of the pituitary gland secretion is obtained. The performed second assessment program obtains, based on the “kind of measuring factor (pituitary gland secretion)” included in the user-inputted required information, the corresponding threshold (pituitary gland secretion) from threshold data stored in a database (S52).

The performed second assessment program compares the blood concentration of a pituitary gland secretion with the corresponding threshold (pituitary gland secretion) (S53) and performs the second assessment. In the case where the pituitary gland secretion is one whose increase in value leads to the assessment that there is an increased risk of the neurodegenerative disease, the assessment that there is a risk of the neurodegenerative disease is made (S54) when the blood concentration is higher than the threshold, in step S53. Alternatively, the assessment that there is no risk of the neurodegenerative disease is made (S55) when the blood concentration is equal to or smaller than the threshold. Once the second assessment result is obtained, determination is performed (FIG. 2(A), S13). In the determination (S13), the processor executes a determination program loaded on a memory from a storage device is executed, thereby realizing a determination unit in a controller.

The performed determination program makes a determination based on the results of the first assessment and second assessment (FIG. 2(A), S13). The controller obtains, based on the “designation of diagnosis mode” included in the user-inputted required information, a corresponding determination table from an association table data of assessment result and determination set beforehand and stored in a database. The determination unit obtains, based on the results of the first assessment and the second assessment and the obtained determination table, the result of determination and produces determination data including the result of determination (FIG. 2(A), S14). An example of the determination table in this example is shown in the table below. Once the determination data is produced, the processor of the controller displays the result of determination in a displaying unit.

TABLE 1

| | | First assessment result | |
|---|---|---|---|
| | | (Risk of neurodegenerative disease) | |
| | | No risk | Risk |
| Second assessment | Risk | (a) Suspected of | (c) Neurodegenerative |
| result (Risk of | | neurodegenerative | disease |
| neurodegenerative | | disease | |
| Disease) | No risk | (b) No findings | (d) Suspected of |
| | | | neurodegenerative |
| | | | disease |

In the aforementioned example, although the diagnosis mode in which it is determined whether the subject has a neurodegenerative disease is designated, the diagnosis mode in which it is determined which of those such as Alzheimer-type dementia a neurodegenerative disease is classified into may be designated. When the diagnosis mode in which it is determined which the disease is classified is designated, the controller obtains, based on the “kind of measuring factor (an inflammatory factor, a pituitary gland secretion, an autonomic nerve secretion, or a brain image) included in user-inputted required information in determination step (FIG. 2(A), S13), a corresponding determination table from association table data of assessment result and determination set beforehand stored in a database. The determination unit obtains the result of determination and produces determination data including the result of determination based on the result of the first assessment and the second assessment and the obtained determination table (FIG. 2(A), S14). An example of the determination table when the measuring factor is an inflammatory factor is shown in the table below.

TABLE 2

| | | First assessment result | |
|---|---|---|---|
| | | (Risk of neurodegenerative disease) | |
| | | No risk | Risk |
| Second assessment | Risk | (a) Suspected of any | (c) Any of |
| result (Risk of any | | of neurodegenerative | neurodegenerative |
| of | | diseases such as | diseases such as |
| neurodegenerative | | Alzheimer-type | Alzheimer-type |
| disease such as | | dementia | dementia |
| Alzheimer-type | No risk | (b) No findings | (d) Suspected of |
| dementia) | | | neurodegenerative |
| | | | disease |

The determination data including the results of the first assessment and the second assessment and the result of determination calculated in the aforementioned example may be stored in a storage device of the determination device, including identification information (for example, the full name, the ID number, the examination day) of the subject. Although the determination data is stored in the storage device built in the determination device in this example, it is not limited thereto. The determination data may be stored in an external storage device connected through an interface to the determination device.

Although the obtainment of the HCA concentration (S31) and the following obtainment of the threshold (HCA) (S32) were performed in this order in the first assessment step (S11) illustrated in the aforementioned example, the diagnostic apparatus for performing the diagnosis-aiding method according to the invention is not limited thereto. In the diagnostic apparatus, for example, the order is not limited as long as the obtainment of the HCA concentration (S31) and the obtainment of the threshold (HCA) (S32) are made before the step (S33) of comparing the HCA concentration with the threshold (HCA) is performed. Accordingly, the obtainment of the HCA concentration and the obtainment of the threshold (HCA) may be made simultaneously or they may be performed in the order of the obtainment of the threshold (HCA) (S32) and then the HCA concentration (S31).

The order of the steps (S51, S52) in the second assessment step (S12) illustrated in the aforementioned example is also not limited, as long as the steps S51 and S52 are performed before the step 53 is performed. Moreover, the order of the first assessment step (S11) and the second assessment step (S12) illustrated in the aforementioned example is also not limited, as long as the steps S11 and S12 are performed before the determination step (S13) is performed. For example, the obtainment step of the HCA concentration (S31), the obtainment step of the threshold (HCA) (S32), the obtainment step of the pituitary gland secretion concentration (S51), and the obtainment step of the threshold (pituitary gland secretion) (S52) may be performed simultaneously, before the determination step (S13) is performed, and then, the assessment step (S33 and S53) may be performed simultaneously.

Examples of embodiments of the invention may include, but are not limited to, the following:

[Item 1] A method for aiding diagnosis of a neurodegenerative disease in a subject, comprising: a first assessment step of assessing a risk of the neurodegenerative disease based on an amount of homocysteine acid in a biological sample obtained from the subject; a second assessment step of assessing a risk of the neurodegenerative disease based on an amount of an inflammatory factor, a pituitary gland secretion, or an autonomic nerve secretion in the biological sample obtained from the subject or based on a measured value obtained from a brain image of the subject (hereinafter, referred to as a “measured value of brain image”); and a step of determining whether the subject has the neurodegenerative disease based on the results of the first assessment and the second assessment.
[Item 2] The method according to item 1, wherein the first assessment step comprises comparing the amount of homocysteine acid in the biological sample with a predetermined threshold and assessing the risk of the neurodegenerative disease based on the result of the comparison.
[Item 3] The method according to item 1 or 2, wherein the second assessment step comprises comparing the amount of an inflammatory factor, a pituitary gland secretion, or an autonomic nerve secretion in the biological sample with a predetermined threshold and assessing a risk of the neurodegenerative disease based on the result of the comparison.
[Item 4] The method according to any of items 1 to 3, wherein the inflammatory factor is C-reactive protein, IL-1β, or TNF-α; the pituitary gland secretion is cortisol or ACTH; or the autonomic nerve secretion is adrenaline or noradrenaline.
[Item 5] The method according to item 1 or 2, wherein the second assessment step comprises comparing the measured value of brain image with a predetermined threshold and assessing a risk of the neurodegenerative disease based on the result of the comparison.
[Item 6] The method according to item 5, wherein the second assessment step comprises assessing that there is a risk of the neurodegenerative disease, when the brain image is a nuclear magnetic resonance image (MRI) and the brain volume obtained from the MRI brain image is smaller than a predetermined threshold or assessing that there is a risk of the neurodegenerative disease when the brain image is an amyloid positron emission tomography (PET) image and the area of amyloid plaques obtained from the amyloid PET image is larger than a predetermined threshold.
[Item 7] The method according to any of items 1 to 6, wherein the determining step comprises determining that the subject has the neurodegenerative disease when both of the results of the first assessment and the second assessment are “there is the risk” of the neurodegenerative disease or determining that the subject is suspected to have the neurodegenerative disease when either of the results of the first assessment and the second assessment are “there is the risk” of the neurodegenerative disease.
[Item 8] The method according to any of items 1 to 7, wherein the neurodegenerative disease is Alzheimer-type dementia.
[Item 9] The method according to item 1 or 2, wherein the determining step comprises determining, based on the results of the first assessment and the second assessment, which of Alzheimer-type dementia, dementia with Lewy bodies, vascular dementia, or frontotemporal dementia the neurodegenerative disease is classified into.
[Item 10] The method according to item 9, wherein the second assessment step comprises assessment based on the measured value of brain image and the determining step comprises determining whether the neurodegenerative disease is classified into frontotemporal dementia based on the results of the first assessment and the second assessment.
[Item 11] The method according to any of items 1 to 10, wherein the biological sample is a blood sample or a urine sample.
[Item 12] A method for aiding diagnosis of a neurodegenerative disease in a subject, comprising: a step of assessing a risk of the neurodegenerative disease based on at least two measured values of measuring factors selected from the group consisting of a measured value of homocysteic acid, a measured value of an inflammatory factor, a measured value of a pituitary gland secretion, a measured value of an autonomic nerve secretion in a biological sample obtained from the subject, and a measured value obtained from a brain image of the subject; and a step of determining whether the subject has the neurodegenerative disease based on the result of the assessment; wherein the at least two measured values of the measuring factors comprise the measured value of homocysteic acid.
[Item 13] The method according to item 12, wherein the assessment step comprises a step of calculating an assessment value from the at least two measured values of the measuring factors and the assessment value comprises comparing the assessment value with a predetermined threshold corresponding thereto and assessing a risk of the neurodegenerative disease based on the result of the comparison.
[Item 14] The method according to item 12 or 13, wherein the inflammatory factor is C-reactive protein, IL-1β, or TNF-α; the pituitary gland secretion is cortisol or ACTH; the autonomic nerve secretion is adrenaline or noradrenaline; the brain image is a nuclear magnetic resonance image (MRI), an amyloid positron emission tomography (PET) image, or computed tomography (CT image).
[Item 15] The method according to any one of items 12 to 14, wherein the at least two measured values of the measuring factors comprise the measured value of homocysteic acid, the measured value of an inflammatory factor, and the measured value of a pituitary gland secretion, the inflammatory factor is TNF-α, and the pituitary gland secretion is cortisol.
[Item 16] The method according to any one of items 12 to 15, wherein the neurodegenerative disease is Alzheimer-type dementia, dementia with Lewy bodies, vascular dementia, or frontotemporal dementia or mild cognitive impairment.
[Item 17] The method according to item 12 or 13, wherein the at least two measured values of the measuring factors comprise the measured value of homocysteic acid and the measured value of an inflammatory factor, the inflammatory factor is TNF-α, and the neurodegenerative disease is mild cognitive impairment.
[Item 18] The method according to any one of items 12 to 17, wherein the biological sample is a blood sample or a urine sample.

Although specific examples are described below, they illustrate preferred embodiments of the invention and do not limit the scope of claims attached hereto in any ways.

### EXAMPLES

### [Participant Subjects and Diagnosis of Dementia]

30 subjects have participated. The 30 subjects were diagnosed according to the diagnosis guidelines such as the Diagnostic and Statistical Manual of Mental Disorders, Fifth Edition (DSM-5) by the American Psychiatric Association and were subjected to the Alzheimer-type dementia and Mini Mental State Examination (MMSE).

As a result of the diagnosis of Alzheimer-type dementia, 9 subjects were diagnosed as the negative control (NC), 10 subjects were diagnosed as mild cognitive impairment (MCI), and 11 subjects were diagnosed as Alzheimer-type dementia (AD).

In the result of MMSE with a perfect score of 30 points, 9 subjects were scored 28 to 30 points, 8 subjects were scored 24 to 27 points, and 13 subjects were scored 23 points or lower. In MMSE, the subjects with 24 to 27 points are assessed as “suspected of MCI” and the subjects with 23 points or lower are assessed as “suspected of dementia”.

### [Measurement of the Amount of Homocysteine Acid]

Peripheral blood aliquots were collected from the 30 subjects and were subjected to centrifugation to obtain the plasma samples as specimens. The amount of homocysteic acid (HCA) in the collected plasma sample was measured by an immunological measurement (enzyme-linked immuno sorbent assay).

Into each well of a microtiter plate, aliquots of a carbonate buffer solution (pH 9.6) containing an HCA-conjugating BSA (bovine serum albumin) were dispensed and the microtiter plate was placed at four degrees Celsius for 48 hours or more to allow HCA-BSA adsorbed onto the microtiter plate. The carbonate buffer solution was removed from the each well. Into the wells, aliquots of a carbonate buffer solution (pH 9.6) containing BSA were dispensed and the microtiter plate was placed at four degrees Celsius overnight for blocking. The carbonate buffer solution was removed from the wells and then a microtiter plate for measurement was prepared by washing the wells twice with PBS-T and drying the wells.

The specimen of 50 μL or a reference solution (homocysteic acid-containing solution at a known concentration) of 50 μL, a diluent of 50 μL, and a labeled antibody solution (solution containing an anti-homocysteic acid antibody labelled with alkaline phosphatase (ALP), that is, an ALP labeled anti-homocysteic acid antibody) of 100 μL were added to a microtest tube and reacted at 37 degrees Celsius for 120 minutes. The reaction solution was dispensed into the wells in the microtiter plate for measurement and reacted at 37 degrees Celsius for 120 minutes. The reaction solution was removed from the wells, and then the wells were washed twice with PBS-T. To the wells of the microtiter plate after the reaction, the photoluminescence substrate CDP-Star was added and then 30 minutes later, the photoluminescence intensity was measured. The HCA concentration in the specimen was calculated from the photoluminescence intensity that was measured from each specimen and the photoluminescence intensity that was measured from the reference solution.

### [Measurement of the Amounts of TNF-α and Cortisol]

The amount of TNF-α and the amount of cortisol in specimens were basically measured using Human TNF-alpha Quantikine HS ELISA (R & D systems, HSTA00E) and cortisol ELISA (Abnova Corporation, KA0918) according to the instructions, respectively. As the amount of TNF-α and the amount of cortisol, the TNF-α concentration and the cortisol concentration in the specimen were calculated from the absorbance of each specimen and the absorbance of the reference solution, respectively.

### [Example 1] Differentiation of Alzheimer-Type Dementia and Negative Control

It was assessed whether negative control (NC) subjects and Alzheimer-type dementia (AD) subjects can be distinguished based on the measured value of homocysteic acid (HCA), the measured value of cortisol, and the measured value of TNF-α in the subjects.

The results of the 30 subjects who were diagnosed as being AD, MCI, or NC and the measured values of HCA, cortisol, and TNFα of each subject were shown below.

TABLE 3

| | | Measured | Measured | Measured | | |
|---|---|---|---|---|---|---|
| | | value of | value of | value of | | |
| Patient | Diagnosis | HCA | cortisol | TNFα | Assessment | |
| No. | result | [pg/mL] | [pg/mL] | [pg/mL] | value | Determination |
| 7 | NC | 0.24 | 87.86 | 1.14 | −0.96 | Negative |
| 12 | NC | 0.86 | 200.28 | 1.09 | −0.17 | Negative |
| 16 | NC | 0.20 | 246.87 | 2.44 | −3.19 | Negative |
| 21 | NC | 0.38 | 95.64 | 1.25 | −1.25 | Negative |
| 23 | NC | 1.69 | 131.88 | 0.89 | −0.46 | Negative |
| 25 | NC | 0.19 | 183.17 | 1.17 | −0.27 | Negative |
| 26 | NC | 0.32 | 117.57 | 1.00 | −0.38 | Negative |
| 27 | NC | 0.43 | 190.14 | 0.60 | 1.24 | Positive |
| 30 | NC | 0.56 | 80.09 | 0.73 | −0.05 | Positive |
| 1 | MCI | 0.17 | 27.87 | 1.64 | −2.79 | Negative |
| 2 | MCI | 0.38 | 180.08 | 2.07 | −2.79 | Negative |
| 3 | MCI | 0.28 | 124.76 | 0.63 | 0.68 | Positive |
| 4 | MCI | 0.17 | 255.53 | 2.12 | −2.24 | Negative |
| 5 | MCI | 0.20 | 191.38 | 1.33 | −0.63 | Negative |
| 8 | MCI | 0.43 | 164.94 | 0.64 | 0.93 | Positive |
| 10 | MCI | 1.16 | 107.08 | 0.91 | −0.53 | Negative |
| 11 | MCI | 0.55 | 270.51 | 1.17 | 0.31 | Positive |
| 24 | MCI | 0.33 | 94.83 | 1.19 | −1.09 | Negative |
| 29 | MCI | 0.70 | 168.53 | 1.00 | −0.11 | Negative |
| 6 | AD | 0.63 | 131.20 | 0.55 | 0.82 | Positive |
| 9 | AD | 0.43 | 127.74 | 0.61 | 0.72 | Positive |
| 13 | AD | 1.29 | 121.62 | 0.77 | −0.09 | Positive |
| 14 | AD | 0.63 | 73.59 | 0.79 | −0.27 | Negative |
| 15 | AD | 1.03 | 112.11 | 0.97 | −0.60 | Negative |
| 17 | AD | 0.13 | 814.09 | 2.29 | 1.81 | Positive |
| 18 | AD | 0.35 | 234.91 | 0.91 | 0.78 | Positive |
| 19 | AD | 0.10 | 180.56 | 0.91 | 0.45 | Positive |
| 20 | AD | 0.33 | 123.28 | 0.72 | 0.43 | Positive |
| 22 | AD | 0.22 | 129.24 | 1.05 | −0.40 | Negative |

The assessment value for determining whether the subject has AD or NC was calculated from the measured values of HCA, cortisol, and TNFα of each subject with use of the following formula.

[Assessment value]=−[HCA]×0.3685+[cortisol]×0.00804−[TNFα]×2.69908+1.48887 [Formula 1]

The assessment values obtained from the subjects are summarized in Table 3. The obtained assessment value of each subject was compared with the threshold and the subject was determined to be positive (being AD) when the assessment value of the subject was larger than the threshold and the subject was determined to be negative (being NC) when the assessment value of the subject was equal to or smaller than the threshold. The results of determination when −0.15 was used as the threshold are summarized in Table 3. The sensitivity and specificity were calculated from the results of determination for each threshold.

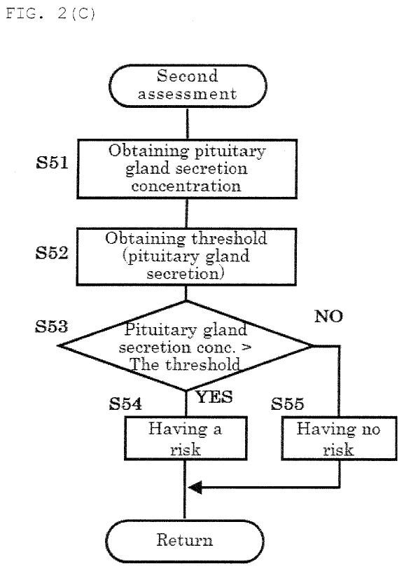

The usefulness of this method for distinguishing AD and NC was assessed based on a receiver operating characteristic (ROC) curve (FIG. 3). The point that is close in distance to the point where (1-specificity:sensitivity) is (0:1) on the ROC curve in FIG. 3 was used as the threshold in this method for distinguishment (◯ in FIG. 3). The sensitivity at the point was 73% (=8/11: 8 subjects was determined to be positive in 11 AD subjects) and the specificity was 78% (=7/9: 7 subjects were determined to be negative in 9 NC subjects).

With the sensitivity and the specificity both being over 70%, this method for distinguishment was assessed to be useful as a method for aiding diagnosis of AD.

### Comparative Examples 1 to 3

The assessment values for determining whether a subject is AD or NC were calculated from the measured values (Table 3) of one of the measuring factors, which is HCA, cortisol, or TNF α of each subject. The usefulness of this method for distinguishment was assessed from an ROC curve, similar to Example 1.

### Comparative Example 1

The sensitivity of the method for distinguishment based on HCA was 45% (=5/11: 5 subjects was determined to be positive in 11 AD subjects) and the specificity was 78% (=7/9: 7 subjects were determined to be negative in 9 NC subjects).

### Comparative Example 2

The sensitivity of the method for distinguishment based on cortisol was 27% (=3/11: 3 subjects was determined to be positive in 11 AD subjects) and the specificity was 67% (=6/9: 6 subjects were determined to be negative in 9 NC subjects).

### Comparative Example 3

The sensitivity of the method for distinguishment based on TNFα was 82% (=9/11: 9 subjects was determined to be positive in 11 AD subjects) and the specificity was 56% (=5/9: 5 subjects were determined to be negative in 9 NC subjects).

With a sensitivity and a specificity both being not over 70%, none of the methods for distinguishment of Comparative Examples 1 to 3 was assessed to be useful as a method for aiding diagnosis of AD.

### [Example 2] Distinguishment Between MMSE of 23 or Lower and MMSE of 28 to 30

It was assessed whether the subjects “suspected of dementia” (MMSE 23 or lower) could be distinguished from the subjects “without any suspect of dementia or MCI” (MMSE 28 to 30) based on the measured values of homocysteic acid (HCA), cortisol, and TNF-α of the subjects.

The MMSE scores of the 30 subjects, and the measured values of homocysteic acid (HCA), cortisol, and TNF-α of the subjects were shown below.

TABLE 4

| | | Measured | Measured | Measured | | |
|---|---|---|---|---|---|---|
| | | value of | value of | value of | | |
| Patient | MMSE | HCA | cortisol | TNF α | Assessment | |
| No. | score | [pg/mL] | [pg/mL] | [pg/mL] | value | Determination |
| 3 | 30 | 0.28 | 124.76 | 0.63 | 0.49 | Positive |
| 10 | 30 | 1.16 | 107.08 | 0.91 | −0.22 | Negative |
| 4 | 29 | 0.17 | 255.53 | 2.12 | −3.12 | Negative |
| 25 | 29 | 0.19 | 183.17 | 1.17 | −0.74 | Negative |
| 26 | 29 | 0.32 | 117.57 | 1.00 | −0.75 | Negative |
| 30 | 29 | 0.56 | 80.09 | 0.73 | −0.14 | Negative |
| 7 | 28 | 0.24 | 87.86 | 1.14 | −1.50 | Negative |
| 24 | 28 | 0.33 | 94.83 | 1.19 | −1.58 | Negative |
| 29 | 28 | 0.70 | 168.53 | 1.00 | −0.12 | Negative |
| 11 | 27 | 0.55 | 270.51 | 1.17 | 0.24 | Positive |
| 15 | 26 | 1.03 | 112.11 | 0.97 | −0.41 | Negative |
| 21 | 26 | 0.38 | 95.64 | 1.25 | −1.73 | Negative |
| 1 | 25 | 0.17 | 27.87 | 1.64 | −3.74 | Negative |
| 8 | 25 | 0.43 | 164.94 | 0.64 | 0.91 | Positive |
| 16 | 25 | 0.20 | 246.87 | 2.44 | −4.23 | Negative |
| 2 | 24 | 0.38 | 180.08 | 2.07 | −3.59 | Negative |
| 5 | 24 | 0.20 | 191.38 | 1.33 | −1.16 | Negative |
| 18 | 23 | 0.35 | 234.91 | 0.91 | 0.65 | Positive |
| 19 | 23 | 0.10 | 180.56 | 0.91 | 0.05 | Positive |
| 13 | 22 | 1.29 | 121.62 | 0.77 | 0.42 | Positive |
| 9 | 21 | 0.43 | 127.74 | 0.61 | 0.66 | Positive |
| 17 | 21 | 0.13 | 814.09 | 2.29 | 1.57 | Positive |
| 6 | 19 | 0.63 | 131.20 | 0.55 | 0.94 | Positive |
| 12 | 18 | 0.86 | 200.28 | 1.09 | −0.06 | Positive |
| 27 | 17 | 0.43 | 190.14 | 0.60 | 1.27 | Positive |
| 20 | 15 | 0.33 | 123.28 | 0.72 | 0.23 | Positive |
| 22 | 15 | 0.22 | 129.24 | 1.05 | −0.85 | Negative |
| 14 | 10 | 0.63 | 73.59 | 0.79 | −0.35 | Negative |
| 28 | 10 | 1.54 | 486.94 | 1.11 | 2.86 | Positive |

The assessment values for determining whether a subject is “suspected of dementia” (MMSE 23 or lower) or “without any suspect of dementia or MCI” (MMSE 28 to 30) were calculated from the measured values of homocysteic acid (HCA), cortisol, and TNF-α of the subjects with use of the following formula.

[Assessment value]=[HCA]×0.39927+[cortisol]×0.00942−[TNFα]×3.22908+1.2428 [Formula 2]

The assessment values obtained from the subjects are summarized in Table 4. The obtained assessment value of each subject was compared with the threshold and the subject was determined to be positive (suspected of dementia) when the assessment value of the subject was larger than the threshold and the subject was determined to be negative (without any suspect of dementia or MCI) when the assessment value of the subject was equal to or smaller than the threshold. The results of determination when −0.10 was used as the threshold are summarized in Table 4. The sensitivity and the specificity were calculated from the results of determination for each threshold.

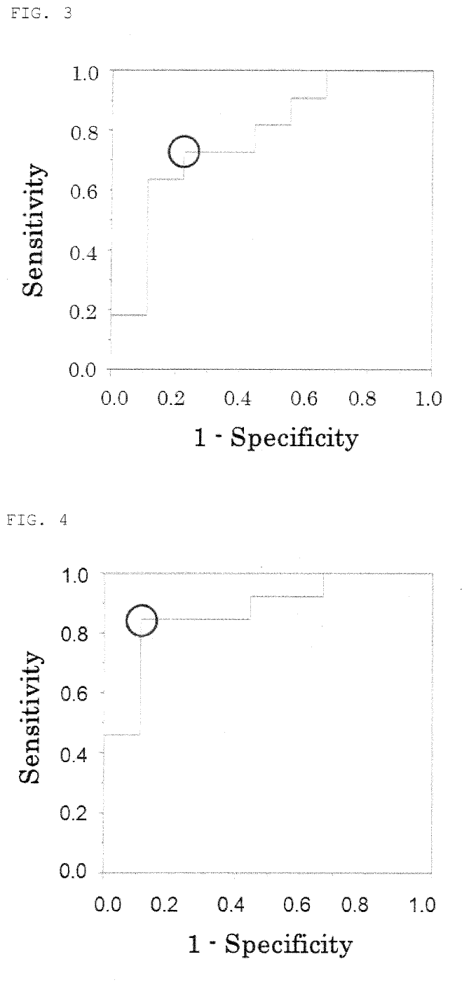

The usefulness of this method for distinguishing “suspected of dementia” and “without any suspect of dementia or MCI” was assessed based on a receiver operating characteristic (ROC) curve (FIG. 4). The point that is close in distance to the point where (1-specificity:sensitivity) is (0:1) on the ROC curve in FIG. 4 was used as the threshold in this method for distinguishment (◯ in FIG. 4). The sensitivity at the point was 85% (=11/13: 11 subjects determined to be positive in 13 subjects with MMSE 23 or lower) and the specificity was 89% (=8/9: 8 subjects determined to be negative in 9 subjects with MMSE 28 to 30).

With the sensitivity and the specificity both being over 80%, this method for distinguishment was assessed to be excellent as a method for aiding diagnosis of dementia.

### Comparative Examples 4 to 6

The assessment values for determining whether a subject is “suspected of dementia” or “without any suspect of dementia or MCI” were calculated from the measured values (Table 4) of one of the measuring factors, which is HCA, cortisol, or TNF α of each subject. The usefulness of this method for distinguishment was assessed from an ROC curve, similar to Example 1.

### Comparative Example 4

The sensitivity of the method for distinguishment based on HCA was 46% (=6/13: 6 subjects determined to be positive in 13 subjects with MMSE 23 or lower) and the specificity was 67% (=6/9: 6 subjects determined to be negative in 9 subjects with MMSE 28 to 30).

### Comparative Example 5

The sensitivity of the method for distinguishment based on cortisol was 39% (=4/13: 4 subjects determined to be positive in 13 subjects with MMSE 23 or lower) and the specificity was 78% (=7/9: 7 subjects determined to be negative in 9 subjects with MMSE 28 to 30).

### Comparative Example 6

The sensitivity of the method for distinguishment based on TNFα was 69% (=9/13: 9 subjects determined to be positive in 13 subjects with MMSE 23 or lower) and the specificity was 56% (=5/9: 5 subjects determined to be negative in 9 subjects with MMSE 28 to 30).

With a sensitivity and a specificity both being not over 70%, none of the methods for distinguishment of Comparative Examples 4 to 6 was assessed to be useful as a method for aiding diagnosis as “suspected of dementia”.

### [Example 3] Comparison with Method for Differential Diagnosis

The sensitivities and specificities of the diagnosis-aiding methods of Example 1 and Example 2 were compared with the sensitivities and specificities of other AD differential diagnosis methods (hTAU in spinal fluid, amyloid PET (¹⁸F), and blood phosphorylated tau).

TABLE 5

| Differential diagnosis method | Sensitivity | Specificity |
|---|---|---|
| Diagnosis-aiding method of Example 1 | 73% | 78% |
| Diagnosis-aiding method of Example 2 | 85% | 89% |
| hTAU in spinal fluid *¹ | 82% | 67% |
| Phosphorylated tau in blood *² | 60% | 85.7% |
| Amyloid PET (¹⁸F) *³ | 90% | 84% |

*¹ Alzheimer's & Dementia: Diagnosis, Assessment & Disease Monitoring 1 (2015) 455-463 (see Table 3 “Total tau”)
*² Molecular Neurodegeneratin (2017) 12: 63 (see FIG. 2)
*³ Eur J Nucl Med Mol Imaging (2016) 43: 374-385 (see Table 6 “Quantitative analysis”).

Any of the methods for differential diagnosis of AD based on the hTAU in spinal fluid and the phosphorylated tau in blood had either a sensitivity or a specificity of less than 70%. On the other hand, the diagnosis-aiding method of Example 1 or 2 was useful in differential diagnosis of a neurodegenerative disease using peripheral blood because of both having a sensitivity and a specificity over 70%. Moreover, the diagnosis-aiding methods of Examples 1 and 2 have the advantage of light physical burdens to subjects in comparison with the method for differential diagnosis of AD based on the hTAU in spinal fluid or amyloid PET (¹⁸F) because it is possible to use peripheral blood for differential diagnosis. The diagnosis-aiding method of Example 2 was a diagnosis-aiding method of a neurodegenerative disease with light physical burdens and with a high sensitivity and a high specificity, which is comparable to conventional methods for distinguishment of AD (amyloid PET (¹⁸F)).

### [Example 4] Distinguishment of MMSE of 24 to 27 and MMSE of 28 to 30

It was assessed whether the subjects “suspected of MCI” (MMSE 24 to 27) can be distinguished from the subjects “without any suspect of dementia or MCI” (MMSE 28 to 30) based on the measured values of homocysteic acid (HCA), cortisol, and TNF-α of the subjects.

The MMSE scores of 17 subjects and the measured values of HCA and TNFα of each subject were shown below.

TABLE 6

| | | HCA | TNFα | Assess- | |
|---|---|---|---|---|---|
| Patient | MMSE | measurement | measurement | ment | |
| No. | score | [pg/mL] | [pg/mL] | value | Determination |
| 3 | 30 | 0.28 | 0.63 | −1.56 | Negative |
| 10 | 30 | 1.16 | 0.91 | 0.19 | Positive |
| 4 | 29 | 0.17 | 2.12 | 1.54 | Positive |
| 25 | 29 | 0.19 | 1.17 | −0.51 | Negative |
| 26 | 29 | 0.32 | 1.00 | −0.71 | Negative |
| 30 | 29 | 0.56 | 0.73 | −0.97 | Negative |
| 7 | 28 | 0.24 | 1.14 | −0.52 | Negative |
| 24 | 28 | 0.33 | 1.19 | −0.28 | Negative |
| 29 | 28 | 0.70 | 1.00 | −0.21 | Negative |
| 11 | 27 | 0.55 | 1.17 | −0.04 | Positive |
| 15 | 26 | 1.03 | 0.97 | 0.15 | Positive |
| 21 | 26 | 0.38 | 1.25 | −0.09 | Positive |
| 1 | 25 | 0.17 | 1.64 | 0.50 | Positive |
| 8 | 25 | 0.43 | 0.64 | −1.35 | Negative |
| 16 | 25 | 0.20 | 2.44 | 2.28 | Positive |
| 2 | 24 | 0.38 | 2.07 | 1.70 | Positive |
| 5 | 24 | 0.20 | 1.33 | −0.15 | Positive |

The assessment values for determining whether a subject is “suspected of MCI” (MMSE 24 to 27) or “without any suspect of dementia or MCI” (MMSE 28 to 30) were calculated from the measured values of HCA, cortisol, and TNF of the subjects with use of the following formula.

[Assessment value]=[HCA]×1.3158+[TNFα]×2.18179−3.31087 [Formula 3]

The assessment values obtained from the subjects are summarized in Table 6. The obtained assessment value of each subject was compared with the threshold and the subject was determined to be positive (suspected of MCI) when the assessment value of the subject was larger than the threshold and the subject was determined to be negative (without any suspect of dementia or MCI) when the assessment value of the subject was equal to or smaller than the threshold. The results of determination when −0.10 was used as the threshold are summarized in Table 6. The sensitivity and the specificity were calculated from the results of determination for each threshold.

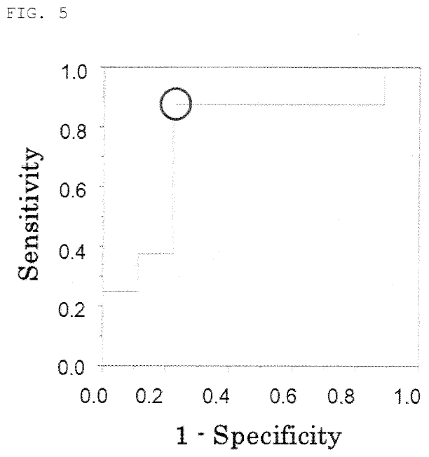

The usefulness of this method for distinguishing “suspected of MCI” and “without any suspect of dementia or MCI” was assessed based on a receiver operating characteristic (ROC) curve (FIG. 5). The point that is close in distance to the point where (1-specificity:sensitivity) is (0:1) on the ROC curve in FIG. 5 was used as the threshold in this method for distinguishment (◯ in FIG. 5). The sensitivity at the point was 88% (=7/8: 7 subjects determined to be positive in 8 subjects with MMSE 24 to 27) and the specificity was 78% (=7/9: 7 subjects determined to be negative in 9 subjects with MMSE 28 to 30).

With the sensitivity and the specificity both being over 75%, this method for distinguishment was assessed to be good as a method for aiding diagnosis of MCI.

The diagnosis-aiding method of Example 4 is a diagnosis-aiding method of a neurodegenerative disease that is capable of distinguishment of MCI, with light physical burdens on subjects.

### Comparative Examples 7 to 9

The assessment values for determining whether a subject is “suspected of MCI” or “without any suspect of dementia or MCI” were calculated from the measured values (Table 6) of one of the measuring factors, which is HCA, cortisol, or TNFα of each subject. The usefulness of this method for distinguishment was assessed based on the ROC curve, similar to Example 1.

### Comparative Example 7

The sensitivity of the method for distinguishment based on HCA was 63% (=5/8: 5 subjects determined to be positive in 8 subjects with MMSE 24 to 27) and the specificity was 33% (=3/9: 3 subjects determined to be negative in 9 subjects with MMSE 28 to 30).

### Comparative Example 8

The sensitivity of the method for distinguishment based on cortisol was 63% (=5/8: 5 subjects determined positive in 8 subjects of MMSE 24 to 27) and the specificity was 67% (=6/9: 6 subjects determined to be negative in 9 subjects with MMSE 28 to 30).

### Comparative Example 9

The sensitivity of the method for distinguishment based on TNFα was 50% (=4/8: 4 subjects determined positive in 8 subjects of MMSE 24 to 27) and the specificity was 89% (=8/9: 8 subjects determined to be negative in 9 subjects with MMSE 24 to 27).

With a sensitivity and a specificity both being not over 70%, none of the methods for distinguishment of Comparative Examples 7 to 9 was assessed to be useful as a method for aiding diagnosis as “suspected of dementia”.

1. A method for aiding diagnosis of a neurodegenerative disease in a subject, comprising:
a first assessment step of assessing a risk of the neurodegenerative disease based on an amount of homocysteine acid in a biological sample obtained from the subject;
a second assessment step of assessing a risk of the neurodegenerative disease based on an amount of an inflammatory factor, a pituitary gland secretion, or an autonomic nerve secretion in the biological sample obtained from the subject or based on a measured value obtained from a brain image of the subject (hereinafter, referred to as a “measured value of brain image”); and
a step of determining whether the subject has the neurodegenerative disease based on the results of the first assessment and the second assessment.

2. The method according to claim 1, wherein the first assessment step comprises
comparing the amount of homocysteine acid in the biological sample with a predetermined threshold and
assessing the risk of the neurodegenerative disease based on the result of the comparison.

3. The method according to claim 1, wherein the second assessment step comprises
comparing the amount of an inflammatory factor, a pituitary gland secretion, or an autonomic nerve secretion in the biological sample with a predetermined threshold and
assessing a risk of the neurodegenerative disease based on the result of the comparison.

4. The method according to claim 1, wherein the inflammatory factor is C-reactive protein, IL-1β, or TNF-α; the pituitary gland secretion is cortisol or ACTH; or the autonomic nerve secretion is adrenaline or noradrenaline.

5. The method according to claim 1, wherein the second assessment step comprises
comparing the measured value of brain image with a predetermined threshold and
assessing a risk of the neurodegenerative disease based on the result of the comparison.

6. The method according to claim 5, wherein the second assessment step comprises
assessing that there is a risk of the neurodegenerative disease, when the brain image is a nuclear magnetic resonance image (MRI) and the brain volume obtained from the MRI brain image is smaller than a predetermined threshold or
assessing that there is a risk of the neurodegenerative disease when the brain image is an amyloid positron emission tomography (PET) image and the area of amyloid plaques obtained from the amyloid PET image is larger than a predetermined threshold.

7. The method according to claim 1, wherein the determining step comprises
determining that the subject has the neurodegenerative disease when both of the results of the first assessment and the second assessment are “there is the risk” of the neurodegenerative disease or
determining that the subject is suspected to have the neurodegenerative disease when either of the results of the first assessment and the second assessment are “there is the risk” of the neurodegenerative disease.

8. The method according to claim 1, wherein the neurodegenerative disease is Alzheimer-type dementia.

9. The method according to claim 1, wherein the determining step comprises determining which of Alzheimer-type dementia, dementia with Lewy bodies, vascular dementia, or frontotemporal dementia the neurodegenerative disease is classified into based on the results of the first assessment and the second assessment.

10. The method according to claim 9, wherein the second assessment step comprises assessment based on the measured value of brain image and the determining step comprises determining whether the neurodegenerative disease is classified into frontotemporal dementia based on the results of the first assessment and the second assessment.

11. The method according to claim 1, wherein the biological sample is a blood sample or a urine sample.

12. A method for aiding diagnosis of a neurodegenerative disease in a subject, comprising:
a step of assessing a risk of the neurodegenerative disease based on at least two measured values of measuring factors selected from the group consisting of a measured value of homocysteic acid, a measured value of an inflammatory factor, a measured value of a pituitary gland secretion, a measured value of an autonomic nerve secretion in a biological sample obtained from the subject, and a measured value obtained from a brain image of the subject; and
a step of determining whether the subject has the neurodegenerative disease based on the result of the assessment;
wherein the at least two measured values of the measuring factors comprise the measured value of homocysteic acid.

13. The method according to claim 12, wherein
the assessment step comprises calculating an assessment value from the at least two measured values of the measuring factors and
the assessment step comprises comparing the assessment value with a predetermined threshold corresponding thereto and assessing a risk of the neurodegenerative disease based on the result of the comparison.

14. The method according to claim 12, wherein the inflammatory factor is C-reactive protein, IL-1β, or TNF-α; the pituitary gland secretion is cortisol or ACTH; the autonomic nerve secretion is adrenaline or noradrenaline; the brain image is a nuclear magnetic resonance image (MRI), an amyloid positron emission tomography (PET) image, or computed tomography (CT image).

15. The method according to claim 12, wherein the at least two measured values of the measuring factors comprise the measured value of homocysteic acid, the measured value of an inflammatory factor, and the measured value of a pituitary gland secretion, the inflammatory factor is TNF-α, and the pituitary gland secretion is cortisol.

16. The method according to claim 12, wherein the neurodegenerative disease is Alzheimer-type dementia, dementia with Lewy bodies, vascular dementia, or frontotemporal dementia or mild cognitive impairment.

17. The method according to claim 12, wherein the at least two measured values of the measuring factors comprise the measured value of homocysteic acid and the measured value of an inflammatory factor, the inflammatory factor is TNF-α, and the neurodegenerative disease is mild cognitive impairment.

18. The method according to claim 12, wherein the biological sample is a blood sample or a urine sample.
